(19) United States
(12) Patent Application Publication
(10) Pub. No.: US 2012/0094963 A1
(43) Pub. Date: Apr. 19, 2012

(54) TARGETING PRODRUGS AND COMPOSITIONS FOR THE TREATMENT OF GASTROINTESTINAL DISEASES

(75) Inventors: John Francis Gilmer, Dublin (IE); Juan Francisco Marquez Ruiz, Malaga (ES); Dermot Kelleher, Dublin (IE)

(73) Assignees: of Queen Elizabeth near Dublin; The Provost Fellows and Scholars of the College of the Holy and Undivided Trinity

(21) Appl. No.: 13/141,663

(22) PCT Filed: Dec. 21, 2009
(86) PCT No.: PCT/EP2009/067689

§ 371 Date: Oct. 12, 2011

Related U.S. Application Data
(60) Provisional application No. 61/140,515, filed on Dec. 23, 2008.

(51) Int. Cl. A61K 31/655; C07D 261/08; A61P 1/00; A61P 29/00; C07D 237/04; A61P 35/00

(52) U.S. Cl. 514/150; 534/664

(57) ABSTRACT

Provided herein are compounds of formula (I) as well as compounds of formula (I) which are prodrugs in which the (R7)a-phenyl-S(O)2NH group represents a sulphonamide-bond compound, compositions and methods for preventing or treating gastrointestinal diseases such as inflammatory bowel disease and colorectal cancer, wherein the method comprises delivering an effective amount of a COX-2 or a similar sulphonamide inhibitor as a prodrug or a derivative thereof to the colon, wherein the COX-2 or similar inhibitor is released in vivo.

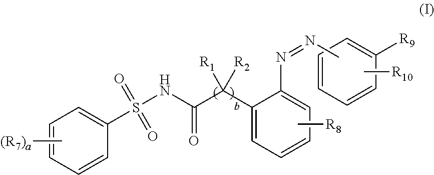

### FIELD OF THE INVENTION

The present application relates to compounds and pharmaceutical compositions that may be used for the treatment and prevention of gastrointestinal diseases including, but not limited to, and cancers such as colorectal and bowel cancer, inflammatory diseases such as Inflammatory Bowel Disease (IBD). In one aspect, the compounds are prodrugs of sulfonamide drugs, particularly but not exclusively COX-2 inhibitors in which the parent COX-2 inhibitor is linked to a carrier. In another aspect, the prodrug compounds have suppressed absorption following oral administration. In yet another aspect, the prodrug may be co-administered with a second therapeutic agent, possibly generating a combination therapy.

### BACKGROUND OF THE INVENTION

Colorectal or bowel cancer is the second leading cause of cancer-related death in the Western world. Incidence is in the range 25-35 per 100,000. It has been suspected for some time that the cyclooxygenase enzymes are involved in the pathophysiology of colon cancer mainly because patients taking inhibitors of the cyclooxygenases (non-steroidal anti-inflammatory drugs (NSAIDs) and aspirin) have a markedly decreased risk of colorectal cancer. The general use of conventional NSAIDS in cancer chemoprevention cannot be recommended because of their gastrointestinal side effect profile.

There are two cyclooxygenase enzymes, COX-1 and COX-2. These have different patterns of expression in humans. The mechanism by which NSAIDs protect against cancer is believed to involve inhibition of the COX-2 isoform. It has been showed that COX-2 expression was elevated in colorectal cancer patients compared to controls. COX-2 appeared to be more important in the early stage of carcinogenesis.

The COX-2 isoform was identified in 1991. The prototype inhibitor for the enzyme DuP697 was reported at around the same time and it exhibited lower gastric toxicity than existing NSAIDs. It became widely accepted that the gastric toxicity of NSAIDs was due to their inhibition of COX-1 in the gastrointestinal tract (GIT) where the enzyme has a gastroprotective role. COX-2 was believed to be primarily induced in response to inflammation. As a result, COX-2 inhibitors were developed in order to act as anti-inflammatory and analgesic agents with lower GI toxicity. It is now apparent that there is a complex interplay between COX-1 and COX-2 in GI protection. However, in large scale clinical trials, selective COX-2 inhibitors have shown lower gastrointestinal toxicity in patients who do not receive aspirin.

A number of important COX-2 selective inhibitors were based on DuP697 and have in common a tripodal structure with a central heterocyclic scaffold bearing two aryl groups, one para substituted with a sulfone or sulfonamide moiety. The latter group is intimately connected with the COX-2 selectivity of the compounds.

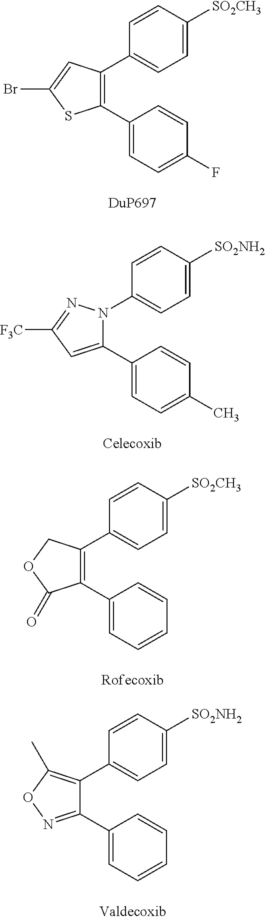

The emergence of the selective COX-2 inhibitors especially celecoxib and rofecoxib permitted pharmacological investigation into the role of COX-2 in colorectal cancer.

Steinbach, et al. (“The effect of celecoxib, a cyclooxygenase-2 inhibitor, in familial adenomatous polyposis.” N. Engl. J. Med. (2000), 342:1946) used celecoxib to treat FAP. In this study 77 patients were enrolled. The treatment groups were similar with regard to race or ethnic group, sex ratio, surgical status, and number of polyps, but they differed in age: the group assigned to 400 mg of celecoxib twice a day was younger (33.1 years) than the group assigned to 100 mg of celecoxib twice a day (38.6 years) and the placebo group (39.9 years). Sixty-six patients had an identified APC mutation, and two additional patients had relatives with known APC mutations. Seventy-two of the 77 patients completed the treatment. More than 90 percent of the patients who completed the study took at least 80 percent of the study drug. At base line, the placebo group had a mean (±SD) of 15.5±13.4 polyps, the group assigned to 100 mg of celecoxib twice a day had a mean of 11.5±8.5 polyps, and the group assigned to 400 mg of celecoxib twice a day had a mean of 12.3±8.2 polyps in the focal areas where polyps were counted (P=0.66 for the comparison among groups).

Treatment with 400 mg of celecoxib twice daily for six months was associated with a significant reduction from base line in the number of colorectal polyps as compared with the placebo group (28.0 percent vs. 4.5 percent, P=0.003). The group receiving 100 mg of celecoxib twice daily had a reduction of 11.9 percent as compared with 4.5 percent in the placebo group (P=0.33). Multivariate linear regression analysis confirmed that 400 mg of celecoxib twice daily reduced the number of colorectal polyps (P=0.005) after adjustment for age, sex, surgical status (colectomy vs. intact colon), number of polyps at base line, and investigational institution. A reduction of 25 percent or more in the mean number of colorectal polyps was seen in 53 percent of the patients in the group receiving 400 mg of celecoxib twice daily (P=0.003 for the comparison with placebo), 31 percent of the patients in the group receiving 100 mg of celecoxib twice daily (P=0.08), and 7 percent of patients in the placebo group. Intention-to-treat analysis of the specific response of rectal polyps as distinct from colonic polyps showed a mean reduction in the number of rectal polyps of 22.5 percent (P=0.01 for the comparison with the placebo group) in the group receiving 400 mg of celecoxib twice daily and of 3.4 percent (P=0.52 for the comparison with the placebo group) in the group receiving 100 mg of celecoxib twice daily, as compared with a mean increase of 3.1 percent in the placebo group. Buchanan, et al. (“Targeting cyclooxygenase-2 and the epidermal growth factor receptor for the prevention and treatment of intestinal cancer.” Cancer Res. (2007), 67:9380) also showed that treatment of Min mice with celecoxib significantly reduced the number of polyps. This study also used an EGFR inhibitor, erlotinib, which also reduced polyp number. However, in combination, these two drugs had a significant additive benefit over single use. Vehicle-treated mice showed an average of 33 small-sized (<1 mm), 37 average sized (1-2 mm), and 25.5 large-sized (>2 mm) small intestinal polyps. Celecoxib as a single agent led to a 60% reduction in the number of <1-mm polyps and an 80% reduction in the number of 1- to 2-mm polyps, whereas no polyps >2 mm in diameter were observed. An overall 78% inhibition of total polyp number was observed in mice treated with celecoxib alone. The use of the EGFR inhibitor erlotinib also had a pronounced effect on the development of small intestinal polyps. Mice that received 50 mg/kg (qd, p.o.) erlotinib showed a 43% and 46% reduction in the number of <1-mm-sized and 1- to 2-mm-sized polyps, respectively. There was also a 96% reduction in the number of large-sized (>2 mm) polyps as well as a 57% inhibition in total polyp number. However, the use of celecoxib and erlotinib in combination had a dramatic effect on the development of small intestinal polyps in APCmin+/mice. This resulted in over a 96% inhibition in the development of small intestinal polyps.

Swamy, et al. (“Chemoprevention of familial adenomatous polyposis by low doses of atorvastatin and celecoxib given individually and in combination to APCMin mice.” Cancer Res (2006), 66:7370) also used combination therapy to treat or prevent polyp formation in min mice. 100 ppm atorvastatin significantly (P<0.002) suppressed intestinal polyp formation when administered in the diet. 300 ppm celecoxib decreased the rate of formation of intestinal polyps by 70% (P <0.0001).

Colorectal cancer risk is clearly reduced in people regularly taking aspirin or COX-2 selective inhibitors. In addition, some epidemiological studies, and most preclinical studies pointed out that specific COX-2 inhibitors like celecoxib are more potent and less toxic than “older” NSAIDs. Twelve carcinogenesis studies support that celecoxib is strikingly potent to prevent intestinal cancer in rats or mice

More recently, it has become apparent that celecoxib can interact with other intracellular components besides its most famous target, cyclooxygenase 2 (COX-2). Even more strikingly, the anticancer effects of celecoxib were also obtained with the use of cancer cell types that do not even contain COX-2.

The VIGOR (Vioxx GI Outcomes Research) study which compared the efficacy and adverse effect profiles of rofecoxib and naproxen revealed a significantly increased risk of heart attack in rofecoxib patients when compared with naproxen patients (0.4% vs. 0.1%, RR 0.25). The difference in overall risk was among patients at higher risk of heart attack, i.e. those meeting the criteria for low-dose aspirin prophylaxis of secondary cardiovascular events (previous myocardial infarction, angina, cerebrovascular accident, transient ischemic attack, or coronary artery bypass). In 2001, Merck commenced the APPROVe (Adenomatous Polyp PRevention On Vioxx) study, a three year trial with the primary aim of evaluating the efficacy of rofecoxib for the prophylaxis of colorectal polyps. An additional aim of the study was to further evaluate the cardiovascular safety of rofecoxib. The APPROVe study was terminated early when the preliminary data from the study showed an increased relative risk of adverse thrombotic cardiovascular events (including heart attack and stroke), beginning after 18 months of rofecoxib therapy. Merck withdrew Vioxx from the market in 2004. In December 2004, “APC,” the first of two trials of celecoxib for colon cancer prevention, found that long-term (33 months) use of high-dose Celebrex (400 and 800 mg daily) demonstrated an increased cardiovascular risk compared with placebo. It is notable that the higher dose is probably required to treat or prevent colorectal cancer. Lower dose celecoxib does not cause an increased risk of heart attack.

It has been concluded that there is a significant increased risk for celecoxib, as well as some other selective and nonselective NSAIDs. However, the increased cardiovascular risk in celecoxib was only noted at high dose. In April 2005, after an extensive review of data, the FDA concluded that it was likely “that there is a ‘class effect’ for increased CV risk for all NSAIDs.” COX-2 selective inhibitors are contra-indicated for individuals at risk of heart attack and carry a ‘black box warning’ in the US.

Systemic administration of selective COX-2 inhibitors is now widely accepted to increase of the risk of heart attack. The chief mechanism proposed to explain the cardiotoxicity is the suppression of prostacyclin, a product of the vascular endothelium which regulates platelet aggregation (Fitzgerald, G. “Coxibs and cardiovascular disease.” N. Engl. J. Med. (2004), 351:1709). Unfortunately, because of the side effects of anti-COX-2 on rates of heart disease, there is no current medical recommendation to use this drug for cancer prevention.

There is therefore a need for a method for delivering COX-2 inhibitors to the colon for cancer chemoprevention and therapy that could minimize or abolish cardiovascular risk.

Delivery of drugs to specific sites in the GI tract has been studied for several reasons. It is convenient to categorize targeted delivery systems into one of four categories: (1) the passage of time (temporal control of delivery), (2) pH-based (triggered by a change in local pH as the formulation passes down the GI tract), (3) enzyme-based (the enzymes found locally in a region of the gut breakdown a prodrug or a formulation to release drug), and (4) pressure-based systems (variations in pressure along the lumen of the GIT is used to trigger drug release).

For delivery of celecoxib to the colon for the treatment of colon cancer, much of the work appears to center on the use of guar gum or chitosan (Haupt, et al. “Luminal delivery and dosing considerations of local celecoxib administration to colorectal cancer.” Eur. J. Pharm. Sci. (2006), 28:204). However, these systems are inefficient.

Thus, COX-2 inhibitors are a class of anti-inflammatory drugs with excellent efficacy and gastrointestinal safety but their use is limited by cardiovascular side effects. These side effects are due mainly to interference in the biochemical machinery of the vascular endothelium which increases the stickiness of vessel wall towards blood components. This cardiovascular liability requires direct contact between the drug and the cells lining vessels in the heart.

On the other hand, it is now widely acknowledged that COX-2 selective inhibitors have a potentially valuable role in preventing colorectal cancer. Their propensity to cause heart attack prevents them from being used for this.

The present application relates to prodrug forms of certain COX-2 inhibitors, and indeed other sulfonamide-bearing drug inhibitors, which may be taken orally. These inhibitors are designed so that they are not absorbed into the blood stream from the oral route. Instead the inhibitors transit to the colon where they are acted upon by colonic microflora releasing the COX-2 or other inhibitor. In one aspect of the design of the present application, the group attached to the COX-2 or other inhibitor (generating the prodrug) is itself also chemopreventative after activation in the colon.

Thus, for example we have been able to couple celecoxib, a well established COX-2 inhibitor to 5-ASA, which is a colonic anti-inflammatory that reduces the risk of cancer. The combination is designed to be activated by colonic microflora with complementary and potentially synergistic effects in cancer prevention.

Drug targeting may be defined as the delivery of a drug to a specific organ, tissue or cell population. This offers the prospect of enhancing the efficacy of drug treatment while reducing systemic impact or side effects. Despite the promise of this approach, and ongoing efforts, there have been few successful examples to date due in part to limited understanding of the basic factors underlying drug transport and the expression of potential targeting vectors. Chemical drug targeting involves the deliberate modification of a drug structure (usually bioreversibly) causing it to accumulate in a target tissue; site-specific release from the prodrug is triggered by a chemical or enzymatic condition not present elsewhere in the body.

The colon is an important challenge to the validity of the drug targeting approach, as conditions in the colon are largely similar to those prevailing elsewhere in the gastrointestinal (GI) system, and the luminal pH gradient through the GI tract is too gradual for effective local drug release on strictly chemical grounds. On the other hand, the colon is an important drug target for the treatment of pathologies of the colon itself, such as inflammatory bowel disease (IBD) and colon cancer, and for the relief of the chronic constipation that accompanies opioid drug treatment. The colon is also important as a potential portal site for peptide and protein drugs that are not absorbed from other regions of the GI tract or are too unstable in the presence of duodenal proteases to be released there

One key difference between the colon and small intestine that might be exploited as a vector for site-specific drug release is the luxuriant microflora of the former. The GI tract has a steadily increasing bacterial concentration gradient on descending from the stomach through the small intestine, followed by an enormous increase at the colon. The bacterial concentration in the small intestine is typically 10³-10⁴ CFU ml⁻¹ whereas the concentration in the colon is 10¹¹-10¹² CFU ml⁻¹ and one third of fecal dry weight consists of bacteria. These organisms fulfil their energy needs by fermenting undigested materials entering from the small intestine (particularly polysaccharides) and have for this purpose evolved an elaborate array of enzymes such as azoreductase, glucosidase, β-glucuronidase, β-xylosidase, nitroreductase, galactosidase and deaminase.

This abrupt increase in bacterial enzyme expression has been investigated as a means of targeting drugs to the colon, especially those for the treatment of IBD. One successful outcome of these endeavours has been the development of azo-based prodrugs of 5-amino salicylic acid (5-ASA) 1 (Scheme 1), which because of their hydrophilicity and polarity pass through the GI system intact before releasing their 5-amino salicylic acid ‘payload’ upon reduction of the azo linker by azoreductases associated with colonic microflora. Several drugs based on this concept, such as ipsalizide, balsalazide 2, sulphasalazine (the prototype) and olsalazide 3, are in clinical use for the treatment of IBD

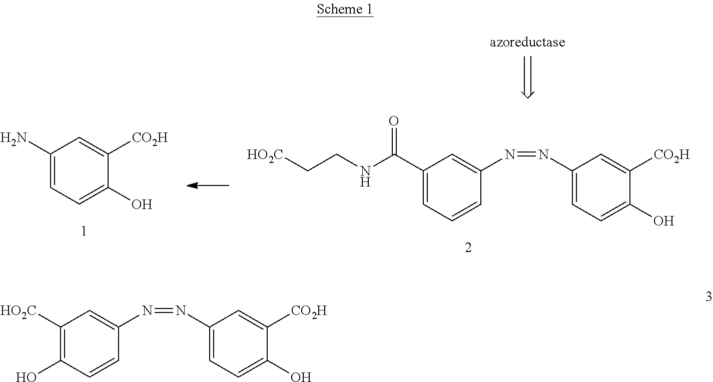

The foregoing examples of the related art and limitations are intended to be illustrative and not exclusive. Other limitations of the related art will become apparent to those of skill in the art upon a reading of the specification and a study of the drawings or figures as provided herein.

### SUMMARY OF THE INVENTION

Provided herein is a novel strategy for drug-targeting sulfonamide-bearing compounds to the colon. The innovative strategy for targeting sulfonamide-bearing drugs to the colon has been designed to overcome the design flaws in the glycosidase targeting approach. One method for achieving the site-specific delivery of amine-bearing drugs is schematically presented in Scheme 2. In one aspect, the drug is selected from the group consisting of an anti-inflammatory drug, and an anti-cancer drug.

In the present application, the inventors discovered a need for compounds, such as the prodrugs disclosed herein, and compositions that are effective as selective agents for COX-2 inhibitory activity and other sulphonamide-bearing compounds and drugs in a patient.

In a first aspect, the present invention provides a compound of the formula

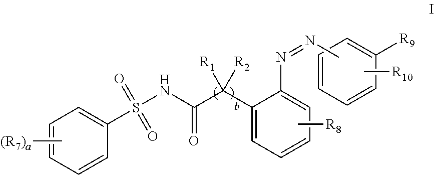

wherein:

each R₁ and R₂ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each alkyl, cycloalkyl, aryl and heteroaryl substituted or unsubstituted; or R₁ and R₂ are together an oxo (═O) group;

each R₃ and R₄ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each alkyl, cycloalkyl, aryl and heteroaryl substituted or unsubstituted; or R₃ and R₄ are together an oxo (═O) group;

each R₅ and R₆ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each alkyl, cycloalkyl, aryl and heteroaryl substituted or unsubstituted; or R₅ and R₆ are together an oxo (═O) group;

R₇ is selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, heteroarylaryl, arylheteroaryl, (C₃₋₁₀)heterocyclyl, amino, carboxy, cyano, halo, hydroxy, sulfamoyl, CONR₁₁R₁₂, (C₁₋₃)alkoxy, aryloxy and heteroaryloxy, each alkyl, cycloalkyl, aryl and heteroaryl substituted or unsubstituted; or alternatively two of R₇, when substituted adjacent on the phenyl ring, together form an aryl, heteroaryl, (C₃₋₁₀)cycloalkyl or (C₃₋₁₀)heterocycloalkyl ring, each substituted or unsubstituted;

each R₈ and R₁₀ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, —SO₃R₁₃, —PO₃R₁₃, (C₁₋₃)alkoxy, aryloxy and heteroaryloxy, each alkyl, cycloalkyl, aryl and heteroaryl substituted or unsubstituted;

R₉ is selected from the group consisting of hydrogen, hydroxy, (C₁₋₃)alkoxy, and —CO₂R₁₃;

or R₉ and R₁₀, when substituted adjacent in the phenyl ring, are taken together to form an optionally substituted heterocyclic ring;

each R₁₁ and R₁₂ is independently selected from the group consisting of hydrogen, (C₁₋₃)alkyl and aryl;

R₁₃ is hydrogen or (C₁₋₃)alkyl;

each a and b is independently 0, 1, 2 or 3;

or a pharmaceutically acceptable salt thereof, optionally in the form of a single stereoisomer or mixture of stereoisomers.

In one embodiment of the present invention, the compound of formula I is a compound of the formula II:

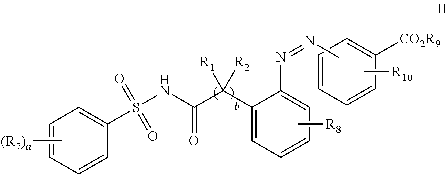

wherein:

R₁, R₂, R₇ and R₈, and a and b, are as defined above;

each R₉ and R₁₀ is independently selected from the group consisting of hydrogen, and (C₁₋₃)alkyl;

or a pharmaceutically acceptable salt thereof, optionally in the form of a single stereoisomer or mixture of stereoisomers.

Preferably, b is 1 or 2 and/or R₉ and R₁₀ are hydrogen in formulae I and II.

In another embodiment of the present invention, the compound of formula I is a compound of the formula III:

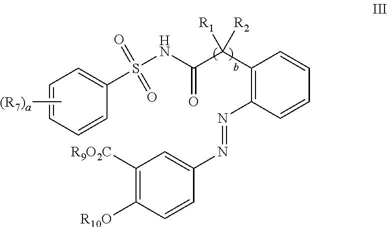

wherein:

each R₁, R₂, R₇, R₉ and R₁₀ and a and b are as defined hereinabove,

or a pharmaceutically acceptable salt thereof, optionally in the form of a single stereoisomer or mixture of stereoisomers.

Preferably, R₁ and R₂ are hydrogen and/or R₉ and R₁₀ are hydrogen in the compounds of formula III.

The compounds of formulae I, II and/or III may be for the treatment of various gastrointestinal diseases, including inflammatory bowel disease (IBD) and/or colorectal cancer.

In another aspect, there is provided a pharmaceutical composition comprising a therapeutically effective amount of a compound or composition of formula I and a pharmaceutically acceptable excipient.

In one aspect, there is provided a compound or composition as defined herein for use in therapy.

In another aspect, there is provided a compound or composition as defined herein for inhibiting COX-2 activity.

In another aspect, there is provided a compound or composition as defined herein for treating cancer and various gastrointestinal diseases, including inflammatory bowel disease (IBD) and colorectal cancer.

In another aspect, there is provided a method comprising administering a therapeutically effective amount of a compound or composition of formula I effective to reduce, alleviate or treat various gastrointestinal diseases, including inflammatory bowel disease (IBD) and/or colorectal cancer.

In another aspect, there is provided a method for inhibiting COX-2 activity in a patient, the method comprising administering a therapeutically effective amount of a compound or composition of formula Ito the patient. Preferably, the therapeutically effective amount is effective to reduce, alleviate, treat or prevent the development of colorectal cancer. The method may also useable where the patient has an increased genetic risk of cancer. The method may also be such that the amount of a compound or composition administered is effective to maintain remission.

In another aspect, there is provided a method comprising administering a therapeutically effective amount of a compound or composition of formula I effective to reduce, alleviate or treat various gastrointestinal diseases, including inflammatory bowel disease (IBD) and/or colorectal cancer, and/or for inhibiting COX-2 activity in a patient, and the co-administration sequentially, simultaneously and/or separately of a therapeutically effective amount of one or more other compounds or compositions able to reduce, alleviate, treat or prevent the development of cancer and/or various gastrointestinal diseases, including inflammatory bowel disease (IBD) and colorectal cancer. Such one or more other compositions may comprise one or more from the group comprising; atorvastatin, valdecoxib, erlotinib and celecoxib.

In another aspect the present invention provides a method for treating gastrointestinal cancer in a mammal, the method comprising delivering a therapeutically effective amount of a COX-2 inhibitor to the colon, wherein the COX-2 inhibitor is released in-vivo from the composition as defined herein.

In particular, the present invention includes and involves compounds being prodrugs wherein the group ‘(R₇)ₐ-phenyl-sulfonamide’ can be or represents the moeity of any active drug compound having a sulfonamide-bearing group or sulfonamide functional group able to link via an amide bond with a carboxy azo carrier as defined herein; that is, any sulfonamide-bonding compound. Such compounds are also termed sulfonamide compounds. Examples of such compounds are mentioned and/or defined herein, including but not limited to COX2 inhibitors. Another example is the amide bonding compound being ethoxzolamide.

The following embodiments, aspects and variations thereof are exemplary and illustrative are not intended to be limiting in scope.

In another aspect, the drug is linked via an amide group to a carrier group, which is connected by an azo-bond to a second carrier group, herein termed a Type-2 prodrug. The carrier groups may be directly attached to the azo group or indirectly attached to the azo group. As provided herein, the carrier groups are designed to maximally suppress absorption from stomach and upper intestine. The method exploits the selective reduction of an azo-linker in the colon, releasing a chemically unstable, latent prodrug that subsequently undergoes cyclization, such as lactamization, that liberates the drug payload, such as a COX-2 inhibitor. For certain compounds, the cyclization reaction is substantially spontaneous. The overall effect of the design is to make the biologically stable or robust amide group, connecting the drug to the carrier group, chemically vulnerable under conditions found only in the colon.

Generally the prodrugs of the present application are referred to as “carrier-drug.” The “carrier” can comprise compounds such as 5-ASA or para-aminobenzoic acid (PABA). When the carrier has a therapeutic effect, such as 5-ASA, the prodrug is generally referred to as a “mutual prodrug.” Such mutual prodrugs can be referred to herein as 5-ASA-drug, wherein the drug can be any appropriate therapeutic agent, including those disclosed herein. Such mutual prodrugs can include, but are not limited to, 5-ASA-ciprofloxacin, 5-ASA-bevacizumab, 5-ASA-prednisolone, 5-ASA-5-ASA, etc. When the carrier does not have a therapeutic effect, such as PABA, the compound can be simply referred to as a “prodrug.” Such prodrugs can be referred to herein as PABA-drug, such as, for example PABA-ciprofloxacin, PABA-bevacizumab, PABA-prednisolone, PABA-5-ASA, etc.

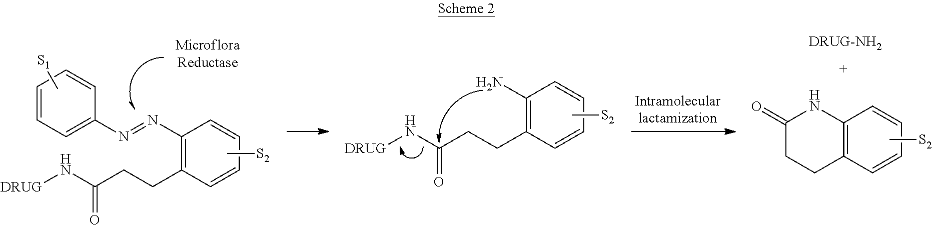

The physicochemical characteristics of the prodrug can be optimized for gastrointestinal penetration to the colon by varying the nature of the compound, including the substituents S₁ and S₂, wherein one or more of the substituents S and S₂ on the aryl ring may be employed, for similar compounds as disclosed herein.

Azo bond reduction proceeds readily because it is based on the promiscuity of the azoreductases present in the colon with respect to substrate, as evidenced by their ability to efficiently reduce substrates as diverse as ipsalazide, in which the carrier group is p-amino hippurate, balsalzide (p-aminobenzoyl-β-alanine carrier), sulfasalazine (sulfapyridine carrier), sterically bulky PAF antagonists (Carceller, et al. “Novel azo derivatives as prodrugs of 5-aminosalicylic acid and amino derivatives with potent platelet activating factor antagonist activity.” J. Med. Chem. (2001), 44, 3001-13), 9-aminocamphothecin (Sakuma, et al. “Biorecognizable HPMA copolymer-drug conjugates for colon-specific delivery of 9-aminocamptothecin.” J. Control Release. (2001), 75, 365-79) and 5-ASA-N-methacrylamide, acryloyloxyethyl and acryloylamido copolymers (e.g. Van den Mooter, et al. “The relation between swelling properties and enzymatic degradation of azo polymers designed for colon-specific drug delivery.” Pharm. Res. (1994), 11, 1737-41). The presence of the vast microflora in the bowel causes a change in redox potential from −67±90 in the distal small bowel to −415±72 in the right colon (Wilding, et al. “Targeting of drugs and vaccines to the gut.” Pharmacol. Ther. (1994), 62, 97-124).

For the Type-2 prodrug, the cyclization or ring closure is dependent on nucleophilic attack by an amine group, such as the aniline group on the carrier. Although anilines possess low nucleophilicity (compared with aliphatic amines) the high effective molarity of the intramolecular arrangement will promote sufficiently rapid ring closure.

Thus, the present invention extends to a process for the delivery of a sulphonamide or sulfonamide-bearing inhibitor as part of a prodrug of formula I as herein defined comprising at least the steps of:

breakage of the azo linkage, to provide the sulfonamide drug and a carrier remnant, and

cyclisation of the resultant carrier remnant.

Gastrointestinal absorption is a function of molecular weight, lipophilicity and polarity; in general, polar, hydrophilic molecules are not well absorbed. Among the possibilities for variation at the carrier group comprising a substituent represented by S_{I}, for example, an azo linkage linking a carrier, including but not limited to 5-ASA, and a second COX-2 inhibitor, thereby generating a mutual prodrug of a drug. Such mutual prodrugs may provide ideal or favourable physicochemical characteristics for passage through the intestine because of mass, polarity and hydrophilicity.

Also included in the above embodiments, aspects and variations are salts of amino acids such as arginate and the like, gluconate, and galacturonate. Some of the compounds of the invention may form inner salts or Zwitterions. Certain of the compounds of the present invention can exist in unsolvated forms as well as solvated forms, including hydrated forms, and are intended to be within the scope of the present invention. Certain of the above compounds may also exist in one or more solid or crystalline phases or polymorphs, the variable biological activities of such polymorphs or mixtures of such polymorphs are also included in the scope of this invention. Also provided are pharmaceutical compositions comprising pharmaceutically acceptable excipients and a therapeutically effective amount of at least one compound of this invention.

Pharmaceutical compositions of the compounds of this invention, or derivatives thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are especially suitable for parenteral administration but may also be used for oral administration. Excipients, such as polyvinylpyrrolidinone, gelatin, hydroxycellulose, acacia, polyethylene glycol, mannitol, sodium chloride, or sodium citrate, may also be added. Alternatively, these compounds may be encapsulated, tableted, or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols, or water. Solid carriers include starch, lactose, calcium sulfate, dihydrate, terra alba, magnesium stearate or stearic acid, talc, pectin, acacia, agar, or gelatin. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulation, and compressing, when necessary, for tablet forms; or milling, mixing, and filling for hard gelatin capsule forms. When a liquid carrier is used, the preparation will be in the form of a syrup, elixir, emulsion, or an aqueous or non-aqueous suspension. Such a liquid formulation may be administered directly p.o. or filled into a soft gelatin capsule.

In one variation, there is provided the above compound, or a pharmaceutically acceptable salt thereof, optionally in the form of a single stereoisomer or mixture of stereoisomers thereof.

In addition to the exemplary embodiments, aspects and variations described above, further embodiments, aspects and variations will become apparent by reference to the drawings and figures and by examination of the following descriptions.

### DETAILED DESCRIPTION OF THE INVENTION

### Definitions

Unless specifically noted otherwise herein, the definitions of the terms used are standard definitions used in the art of organic synthesis and pharmaceutical sciences. Exemplary embodiments, aspects and variations are illustrated in the figures and drawings, and it is intended that the embodiments, aspects and variations, and the figures and drawings disclosed herein are to be considered illustrative and not limiting.

An “alkyl” group is a straight, branched, saturated or unsaturated, aliphatic group having a chain of carbon atoms, optionally with oxygen, nitrogen or sulfur atoms inserted between the carbon atoms in the chain or as indicated. A (C₁-C₂₀)alkyl, for example, includes alkyl groups that have a chain of between 1 and 20 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, penta-1,4-dienyl, hexa-1,3-dienyl, hexa-1,3,5-trienyl, and the like. An alkyl group may also be represented, for example, as a —(CR¹R²)ₘ— group where R¹ and R² are independently hydrogen or are independently absent, and for example, m is 1 to 8, and such representation is also intended to cover both saturated and unsaturated alkyl groups.

An alkyl as noted with another group such as an aryl group, represented as “arylalkyl” for example, is intended to be a straight, branched, saturated or unsaturated aliphatic divalent group with the number of atoms indicated in the alkyl group (as in (C₁-C₂₀)alkyl, for example) and/or aryl group (as in (C₅-C₁₋₄)aryl, for example) or when no atoms are indicated means a bond between the aryl and the alkyl group. Nonexclusive examples of such group include benzyl, phenethyl and the like.

An “alkylene” group is a straight, branched, saturated or unsaturated aliphatic divalent group with the number of atoms indicated in the alkyl group; for example, a —(C₁-C₃)alkylene- or —(C₁-C₃)alkylenyl-.

An “aryl” group is a monocyclic or bicyclic aromatic hydrocarbon group having 5 to 8 atoms in the ring, such as a phenyl. The monocyclic aryl groups are typically are 5 to 7 membered rings, and the bicyclic aryl groups are typically 7 to 8 membered rings.

The term “heteroaryl,” as used herein, means an aryl group containing from, for example, about 3 to about 30 atoms, preferably from about 6 to about 18 atoms, more preferably from about 6 to about 14 atoms, and most preferably from about 6 to about 10 atoms and from 1 to 3 heteroatoms (e.g., N, O or S). Examples of such groups include pyrrolyl, imidazolyl, pyrazolyl, furanyl, oxazolyl, isooxazolyl, thiofuranyl, thiazolyl, isothiazolyl, indolyl, isoindolyl, benzofuranyl, quinolinyl, pyridinyl, pyridazinyl, pyrazinyl, triazolyl and benzotriazolyl.

A “cyclyl” such as a monocyclyl or polycyclyl group includes monocyclic, or linearly fused, angularly fused or bridged polycycloalkyl, or combinations thereof. Such cyclyl group is intended to include the heterocyclyl analogs. A cyclyl group may be saturated, partially saturated or aromatic.

“Halogen” or “halo” means fluorine, chlorine, bromine or iodine.

A “heterocyclyl” or “heterocycle” is a cycloalkyl wherein one or more of the atoms forming the ring is a heteroatom that is a N, O, or S, Non-exclusive examples of heterocyclyl include piperidinyl, 4-morpholinyl, 4-piperazinyl, pyrrolidinyl, 1,4-diazaperhydroepinyl, 1,3-dioxanyl, and the like.

The term “alkoxy” includes linear or branched alkyl groups that are attached to divalent oxygen. The alkyl group is as defined above. Examples of such substituents include methoxy, ethoxy, t-butoxy, and the like. The term “alkoxyalkyl” refers to an alkyl group that is substituted with one or more alkoxy groups. The term “heteroaryloxy” refers to a heteroaryl group that is substituted with one or more alkoxy groups. The term “aryloxy” refers to an aryl group that is attached to an oxygen, such as phenyl-O—, etc.

As used herein, where a divalent group is represented by a group —Z— as described herein, or generically as -A-B—, as shown below for example, it is intended to also represent a group that may be attached in both possible permutations, as noted in the two structures below.

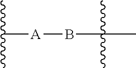

may also be

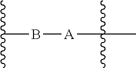

For example, when a divalent group such as the group “—NR′C(O)—” is provided, for example, the group is intended to also include both the divalent group —NR′C(O)— and also the divalent group —C(O)NR′—.

“Pharmaceutically acceptable salts” means salt compositions that is generally considered to have the desired pharmacological activity, is considered to be safe, non-toxic and is acceptable for veterinary and human pharmaceutical applications. Such salts include acid addition salts formed with inorganic acids such as hydrochloric acid, hydrobromic acid, sulfuric acid, phosphoric acid, and the like; or with organic acids such as acetic acid, propionic acid, hexanoic acid, malonic acid, succinic acid, malic acid, citric acid, gluconic acid, salicylic acid and the like.

“Pro-drug” or “prodrug” as used herein, means a bioprecursor or pharmaceutically acceptable compound that may be convertible or degradable in the body, specifically in the colon, to produce a biologically active compound(s) of the invention (for example, the intermediate aniline or the lactam and the active drug). In particular, the compounds of the present application may be reduced by an in vivo azoreductase such as microflora azoreductase.

“Therapeutically effective amount” means a drug amount that elicits any of the biological effects listed in the specification.

“Substituted or unsubstituted” or “optionally substituted” means that a group such as, for example, alkyl, aryl, heterocyclyl, (C₁-C₈)cycloalkyl, heterocyclyl(C₁-C₈)alkyl, aryl(C₁-C₈)alkyl, heteroaryl, heteroaryl(C₁-C₈)alkyl, and the like, unless specifically noted otherwise, may be unsubstituted or, may substituted by 1, 2 or 3 substituents selected from the group such as halo, nitro, trifluoromethyl, trifluoromethoxy, methoxy, carboxy, —NH₂, —OH, —SH, —NHCH₃, —N(CH₃)₂, —SMe, cyano and the like.

### EXPERIMENTAL

### General

Celecoxib is a selective COX-2 inhibitor drug used as an anti-inflammatory. Prodrugs of sulfonamide compounds like celecoxib can be synthesised based on using the sulfonamide group together with the carboxylic acid group from an azo carrier molecule to form an acylsulfonamide group for example, following a condensation reaction. Deprotection of protective groups such as a tert-butyl group can be simply carried out for example using trifluoroacetic acid in DCM at room temperature to yield the prodrugs of the present invention.

The following procedures may be employed for the preparation of exemplary compounds of the present application. The starting materials and reagents used in preparing these compounds are either available from commercial suppliers such as the Sigma Aldrich Chemical Company (Milwaukee, Wis.), Bachem (Torrance, Calif.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In some cases, protective groups may be introduced and finally removed. Suitable protective groups for amino, hydroxy, and carboxy groups are described in Greene et al., Protective Groups in Organic Synthesis, Second Edition, John Wiley and Sons, New York, 1991. Standard organic chemical reactions can be achieved by using a number of different reagents, for examples, as described in Larock: Comprehensive Organic Transformations, VCH Publishers, New York, 1989.

The compounds of this application can be prepared by the steps outlined in the Schemes below.

General Experimental Procedures

Uncorrected melting points were obtained using a Stuart® melting point SMP11 melting point apparatus. Infra-red (IR) spectra were obtained using a Perking Elmer 205 FT Infrared Paragon 1000 spectrometer. Band positions are given in cm⁻¹. Solid samples were obtained by KBr disk; oils were analyzed as neat films on NaCl plates. UV spectroscopy was performed on a Cary 3E UV-VIS spectrophotometer. ¹H and ¹³C spectra were recorded at 27° C. on a Bruker DPX 400 MHz FT NMR spectrometer (400.13 MHz ¹H, 100.16 MHz ¹³C), in either CDCl₃ or CD₃OD, (tetramethylsilane as internal standard). For CDCl₃, ¹H NMR spectra were assigned relative to the TMS peak at 0.00 δ and ¹³C NMR spectra were assigned relative to the middle CDCl₃ triplet at 77.00 ppm. For CD₃OD, ¹H and ¹³C spectra were assigned relative to the centre peaks of the CD₃OD multiplets at 3.30 δ and 49.00 ppm respectively. Coupling constants were reported in hertz (Hz). For ¹H NMR assignments, chemical shifts are reported: shift values (number of protons, description of absorption (s=singlet, d=doublet, t=triplet, q=quartet, m=multiplet), coupling constant(s) where applicable, proton assignment). LRMS was performed on a micromass mass spectrophotometer (EI mode) at the Department of Chemistry, Trinity College. Flash chromatography was performed on Merck Kieselgel 60 particle size 0.040-0.063 mm thin layer chromatography (TLC) for which Rf values are quoted, was performed on silica gel Merck F-254 plates. Compounds were visually detected by absorbance at 254 nm+/or vanillin staining.

Sulfonamide COX-2 Inhibitor Prodrug Synthesis:

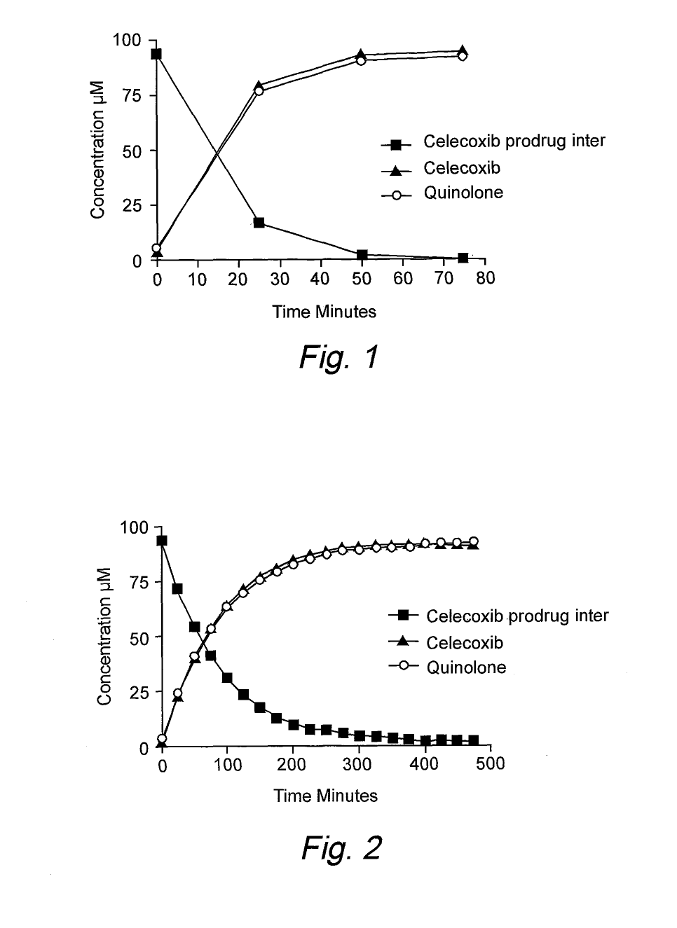

In one embodiment, the present application provides prodrug forms of sulfonamido-based COX-2 inhibitor drugs such as celecoxib and valdecoxib. The prodrug forms are designed to suppress intestinal absorption but are activated by the colonic microflora releasing the COX-2 inhibitor locally, minimizing cardiovascular side effects. In the prodrug design, the carrier group modifies the physicochemical characteristics of the parent suppressing absorption in the upper GIT. Drug release in the colon is triggered in each case by colonic bacteria. We have also found that it is feasible to use a carrier group, which is itself pharmacologically active, and incorporate the carrier group into the drug. For example, 5-amino salicylic acid which has synergistic chemopreventative effects with COX-2 inhibition, may also be incorporated into the prodrug, as illustrated in the FIG. 1 below.

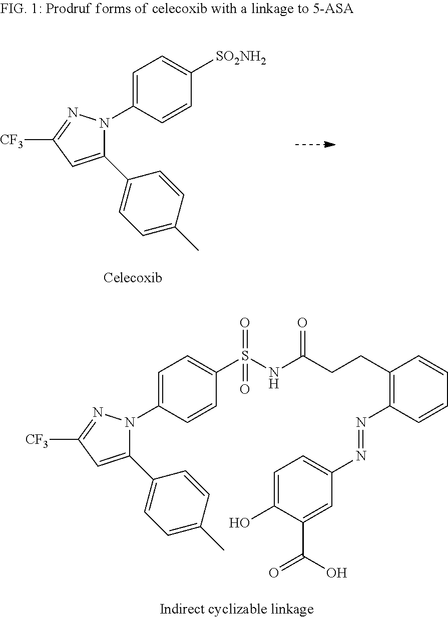

Type 2 prodrugs are activated by azoreductase activity releasing the carrier (here, using 5-ASA) and an amino-ester intermediate which is poised to undergo spontaneous cyclization releasing quinolone.

Activation of Prodrug Type 2:

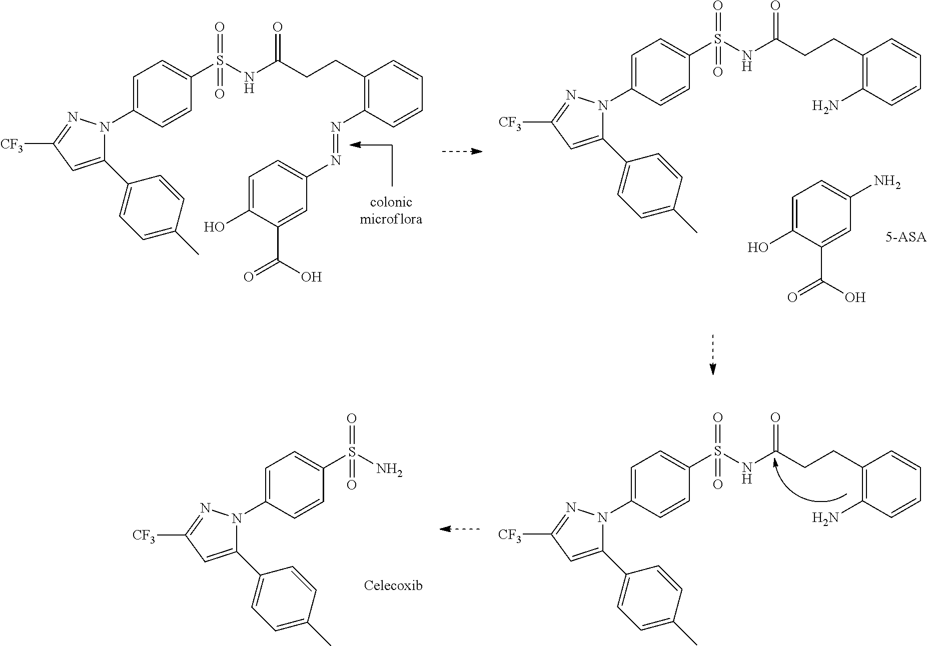

Other hydrophilic carriers that may also be used as an alternative to 5-ASA include 4-ASA and para-aminobenzoic acid.

Synthesis:

Type 2 Compound Synthesis:

In the Type 2 compounds, an amide bond links the COX-2 or other sulfonamide inhibitor with the carrier. Various sulfonamides may be used, including, for example, ethoxzolamide. Acylation of sulfonamides can be accomplished by a number of classical and modern techniques known in the art. The coupling proceeds in good yield using the coupling agent EDC on a solid polymer support. The acyl component (linker) is generated using chemistry as disclosed below. As exemplified below, the salicylic acid portion of the carrier group is protected as a tert-butyl ester.

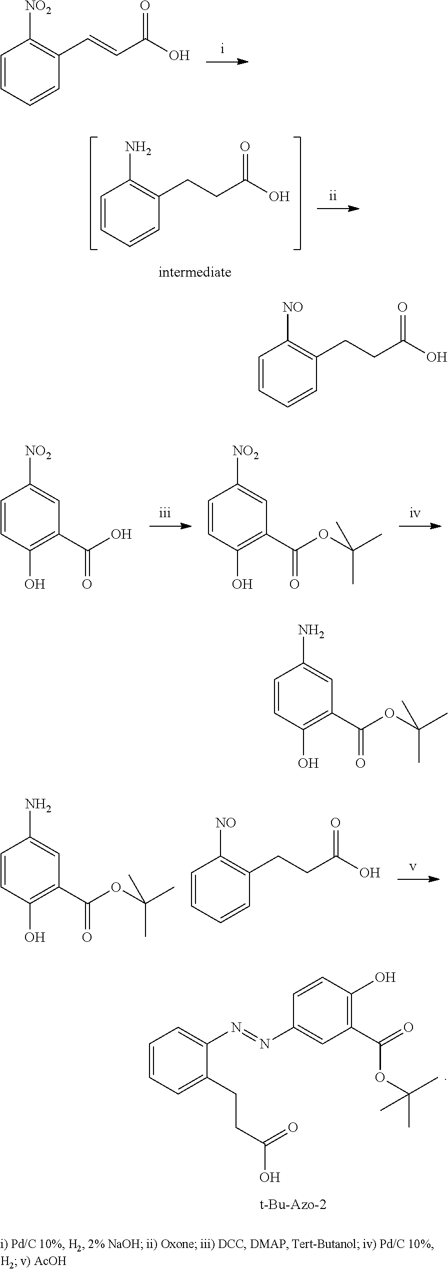

### Example 1

### Celecoxib Prodrug Analogue (PC1) Using 5-ASA

### 5-Nitrosalicylic acid dioxin-4-one

5-Nitrosalicylic acid (20 g, 109 mmol) was placed in a 500 ml round bottom flask equipped with a magnetic stirrer and a reflux condenser, 200 ml of trifluoroacetic acid was added followed by trifluoroacetic anhydride (45.5 ml, 328 mmol) and dry acetone (16.0 ml, 218 mmol). The reaction mixture was left at reflux for two hours. A further 8.02 ml of dry acetone was dropped to the boiling solution every hour (1 eq. per hour) until reaction is complete within eight hours. The reaction is then concentrated under reduced pressure at about 55° C. bath temperature, toluene was added and remove three times to eliminate any trifluoroacetic acid traces and finally the solid residue was dried under vacuum for one hour at 45° C. The crude brownish solid was recrystallized twice from a mixture of acetone-petroleum ether (1:4), to yield 20.5 g of off-white crystals (84.1%). (Alternatively flash chromatography can be used to purify the product when smaller scales are involved, using acetone-petroleum ether (1:4) or dichloromethane as a mobile phase): m.p. 92-94° C., IRᵥₘₐₓ(KBr): 1738.92 (C═O), 1616.23 and 745.94 (NO₂) cm⁻¹.

¹H NMR δ (CDCl₃): 8.88 (1H, d, J=2.51 Hz), 8.46 (1H, dd, J 6.52 and 3.01 Hz), 7.16 (1H, d, J=9.53 Hz), 1.80 (6H, s, 2×CH₃).

¹³C NMR δ (CDCl₃): 159.82 (C, C-7, C═O), 158.50 (C, C-4), 142.33 (C, C-1), 130.79 (CH, C-6), 125.57 (CH, C-2), 118.09 (CH, C-5), 112.93 (C, C-3), 107.36 (C, C-8), 25.45 (CH₃, C-9 and C-10).

### Acetonide-protected 5-aminosalicylic acid

To a solution of 5-nitrosalicylic acid dioxin-4-one (2 g, 8.96 mmol) in ethyl acetate (30 ml) at room temperature, was added palladium 10% on activated charcoal (0.5 g). The mixture was stirred under hydrogen atmosphere until reaction completed by TLC dichloromethane as mobile phase. The palladium was filtered through filter agent and the solvents removed under reduced pressure to yield 1.44 g of product as yellow crystals (83.2%): m.p. 122-124° C., IR_{Vmax}(KBr): 1710.37 (C═O), 3469.02 and 1324.22 (NH₂) cm⁻¹. MS: 216.0616 (M⁺⁺²³), 194.0814 (M⁺⁺¹).

¹H NMR δ (CDCl₃): 7.24 (1H, d, J=3.01 Hz), 6.91 (1H, dd, J 5.52 and 3.01 Hz), 6.79 (1H, d, J=9.04 Hz), 3.61 (2H, s, NH₂), 1.69 (6H, s, 2×CH₃).

¹³C NMR δ (CDCl₃): 161.16 (C, C-7, C═O), 148.15 (C, C-4), 141.36 (C, C-1), 123.38 (CH, C-6), 117.41 (CH, C-2), 113.54 (CH, C-5), 113.51 (C, C-3), 105.75 (C, C-8), 25.20 (CH₃, C-9 and C-10).

### 2-(2,2-Dimethyl-4-oxo-4H-benzo[1,3]dioxin-6-ylazo)-phenyl]-acetic acid (acetonide protected carrier):

To a solution of 2-nitrosophenyl acetic acid (0.5 g, 2.58 mmol) in glacial acetic acid (25 ml) another solution of 5-aminosalicylic acid dioxin-4-one (0.42 g, 2.58 mmol) in glacial acetic acid (25 ml) was added. The reaction mixture was stirred vigorously for 48 h under a nitrogen atmosphere; reaction completion was checked by TLC using dichloromethane-ethyl acetate (50:50) as mobile phase. The solvent was removed under reduced pressure and toluene added twice, followed by evaporation, to eliminate any of acetic acid traces to afford an orange crude product. This was purified by flash chromatography using dichloromethane-ethyl acetate (70:30) as mobile phase to yield 0.78 g of product as orange crystals (89%): m.p. 124-126° C., IR_{Vmax}(KBr): 1734.84 and 1698.18 (C═O) cm⁻¹. MS: 363.0950 (M⁺⁺²³).

¹H NMR δ (CDCl₃): 8.52 (1H, d, J=2.51 Hz), 8.05 (1H, dd, J 8.54 and 2.51 Hz), 7.77 (1H, d, J=7.53 Hz), 7.47 (1H, t, J=6.57 Hz), 7.43 (2H, m), 7.05 (1H, d, J=9.03 Hz), 4.16 (s, 2H), 1.80 (6H, s, 2×CH₃).

¹³C NMR δ (CDCl₃): 177.67 (C, C-8, C═O), 160.42 (C, C-15, C═O), 157.85 (C, C-12), 149.66 (C, C-1), 147.86 (C, C-9), 133.78 (C, C-2), 131.55 (CH, C-4), 131.45 (CH, C-3), 128.93 (CH, C-5), 128.39 (CH, C-14), 126.64 (CH, C-10), 118.14 (CH, C-6), 116.07 (CH, C-13), 113.61 (C, C-11), 106.90 (C, C-16), 37.28 (CH₂, C-7), 25.86 (CH₃, C-17 and C-18).

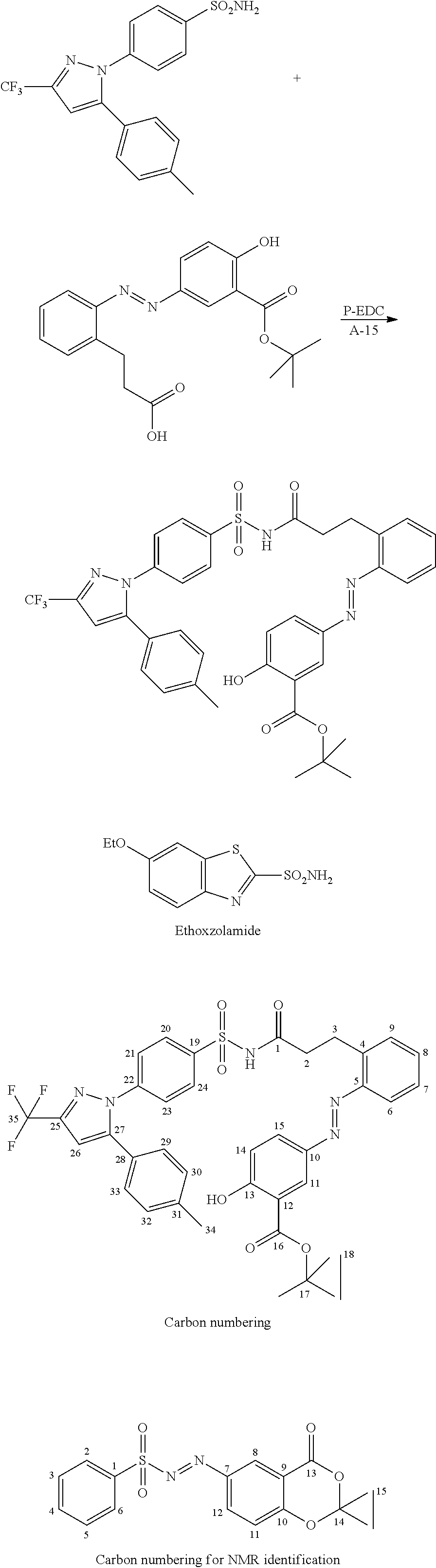

Benzenesulfone and 5-Nitroso Salicylic Acid Acetonide Azo Coupling Product

Benzenesulfonamide (0.2 g, 1.27 mmol) was dissolved in acetic acid (2 ml) and added into a solution of 5-nitroso salicylic acid acetonide (0.26 g, 1.27 mmol). The reaction mixture was left stirring in the microwave reactor at 160° C., with a power of 140 watts under the high absorption mode for 1 h. TLC analysis showed disappearance of the starting material. After removal of the solvent under reduced pressure a flash chromatography was performed using methylene chloride as mobile phase to yield the product as yellow crystals (0.05 g, 11.4%). 3438.06, 1749.34, 1614.94, 1438.75 cm⁻¹.

¹H-NMR (CDCl₃) δ: 8.98 (2H, dd, J 8.8 and 2.28 Hz), 8.56 (1H, dd, J 8.76 and 2.52 Hz), 8.43 (1H, dd, J 8.8 and 2.28 Hz), 8.18 (1H, dd, J 8.76 and 2.48 Hz), 7.13 (3H, m), 1.80 (6H, s).

¹³C-NMR (CDCl₃) δ: 160.52 (C, C═O, C-13), 159.80 (C-10), 142.74 (C, C-1), 138.77 (C, C-7), 134.37 (CH, C-4), 130.14 (CH, C-12), 127.07 (C-3), 125.12 (CH, C-5), 124.11 (CH, C-8), 118.19 (CH, C-2), 117.97 (CH, C-6), 117.65 (CH, C-11), 113.31 (CH, C-9), 106.99 (C, C-14), 25.96 (CH₃, C-15).

Tert-Butyl Ester of Celecoxib Azocarrier Prodrug

Into a round bottom flask was added the azo carrier (0.35 mmol, 0.13 g), t-BuOH (4.5 ml), ClCH₂CH₂Cl (4.5 ml), DMAP (3 eq., 1.05 mmol, 0.13 g), P-EDC (1.69 g) and Celecoxib (0.7 eq., 0.25 mmol, 0.09 g). The resulting mixture was allowed to stir at room temperature for 24 hours. The reaction mixture was then diluted with EtOAc (4 ml), and Amberlyst-15 (2.6 g) was added to the reaction and the reaction was left stirring for additional two hours. At this time the reaction was filtered through a short plug of silica gel and the residue was rinsed with additional EtOAc. The resulting filtrate was concentrated to afford the product as orange oil (0.16 g). The crude mixture was columned using methylene chloride:EtOAc (9:1) as a mobile phase to yield the product as orange oil (70%).

IRᵥₘₐₓ(KBr): 3071.79, 1722.97, 1672.76, 1594 cm⁻¹.

¹H-NMR (CDCl₃) δ: 11.51 (1H, s), 8.42 (1H, d, J=2.48 Hz), 7.98 (1H, dd, J 9.04 and 2.52 Hz), 7.93 (2H, d, J=9.04 Hz), 7.65 (1H, m), 7.46 (2H, d, J=8.52 Hz), 7.32 (1H, m), 7.20 (3H, m), 7.09 (4H, m), 6.76 (1H, s), 3.37 (2H, t, J=7.52 Hz), 2.69 (2H, t, J=7 Hz), 2.40 (3H, s), 1.68 (9H, s).

¹³C-NMR (CDCl₃) δ: 169.01 (C, C═O, C-16), 164.04 (C, C-13), 149.40 (C, C-5), 144.86 (C, C-27), 144.82 (C, C-10), 143.99 (C, C-25), 143.60 (C, C-35), 142.99 (C, C-22), 139.44 (C, C-4), 137.91 (C, C-19), 137.14 (C, C-28), 130.71 (C, C-28), 130.04 (CH, C-9), 129.39 (CH, C-21), 129.02 (CH, C-23), 128.98 (CH, C-30, C-32), 128.65 (CH, C-11), 128.25 (CH, C-7), 127.32 (CH, C-29, C-33), 126.21 (CH, C-15), 125.19 (C, C-31), 124.58 (CH, C-20, C-24), 118.23 (CH, C-14), 115.45 (CH, C-6), 113.46 (C, C-12), 106.07 (CH, C-26), 83.39 (C, C-17), 38.01 (CH₂, C-2), 27.75 (CH₃, C-18), 25.72 (CH₂, C-3), 20.89 (CH₃, C-34).

### 2-Hydroxy-5-(2-3{-oxo-3[4-(5-p-tolyl-3-trifluoromethyl-pyrazol-1-yl)-benzenesulfonylamino]-propyl}-phenyl azo)-benzoic acid (Celecoxib Prodrug PC1)

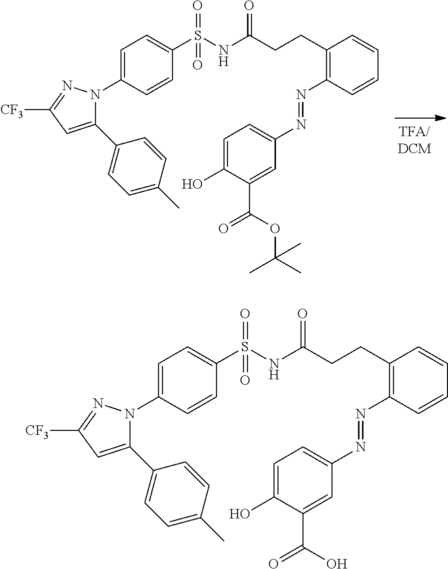

The tert-butyl ester of the Celecoxib (0.16 g) was dissolved in methylene chloride/TFA 1:1 (2 ml) at room temperature for four hours, until the TLC analysis (EtOAc) showed reaction completion. The solvent was evaporated by nitrogen blow and the crude mixture was purified by column chromatography to yield the product as orange crystals (0.13 g, 88%), m.p. over 260° C.

IRᵥₘₐₓ(KBr): 3434.84, 2923.82, 1642.30, 1095.99 cm⁻¹.

¹H-NMR (DMSO) δ: 9.55 (s, 1H, NH), 8.32 (s, 1H), 7.97 (s, 1H), 7.84 (d, 1H, J=5.8 Hz), 7.56 (d, 1H J 5.24 Hz), 7.44 (d, 2H, J=5.04 Hz), 7.36 (m, 2H), 6.80 (d, 1H, J=6.04 Hz), 6.62 (d, 1H, J=5.8 Hz), 6.11 (s, 1H), 2.64 (t, 2H J 5.24 Hz).

¹³C-NMR (DMSO) δ: 169.90 (C, C═O, C-16), 150.18 (C, C-5), 140.01 (C, C-4), 130.61 (C, C-28), 130.11 (CH, C-9), 127.19, 125.97, 124.84, 122.48, 119.56, 118.10, 115.78, 115.78, 115.77 (CH, C-14), 115.37 (CH, C-6), 107.50 (CH, C-26), 38.37 (CH₂, C-2), 27.17 (CH₂, C-3), 21.06 (CH₃, C-34).

HRMS: Expected (M-H⁺)=678.16287, Found (M-H⁺)=678.16284

### Example 2

### Celecoxib Prodrug Analogue (PC2) Using PABA Instead of 5-ASA

Attaching a different carrier to celecoxib yielded an analogue of PC1 hereinafter PC2. 4-aminobenzoic acid (PABA) was used, and the azo group was formed attaching the amino group and the nitroso of the 2-nitrosophenylpropionic acid. The scheme of the PC2 synthesis is described hereinafter.

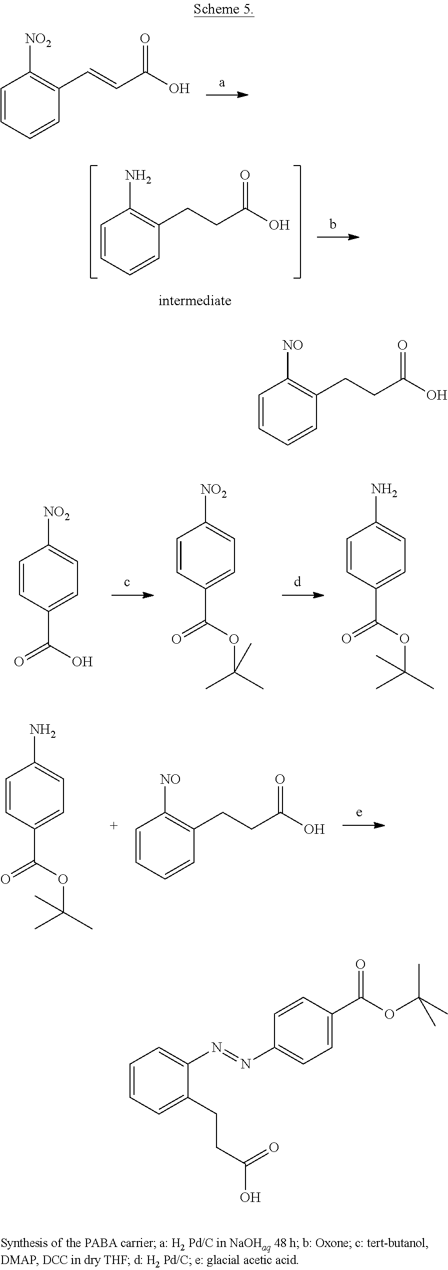

The coupling to celecoxib was carried out in the same synthetic pathway previously used for PC1.

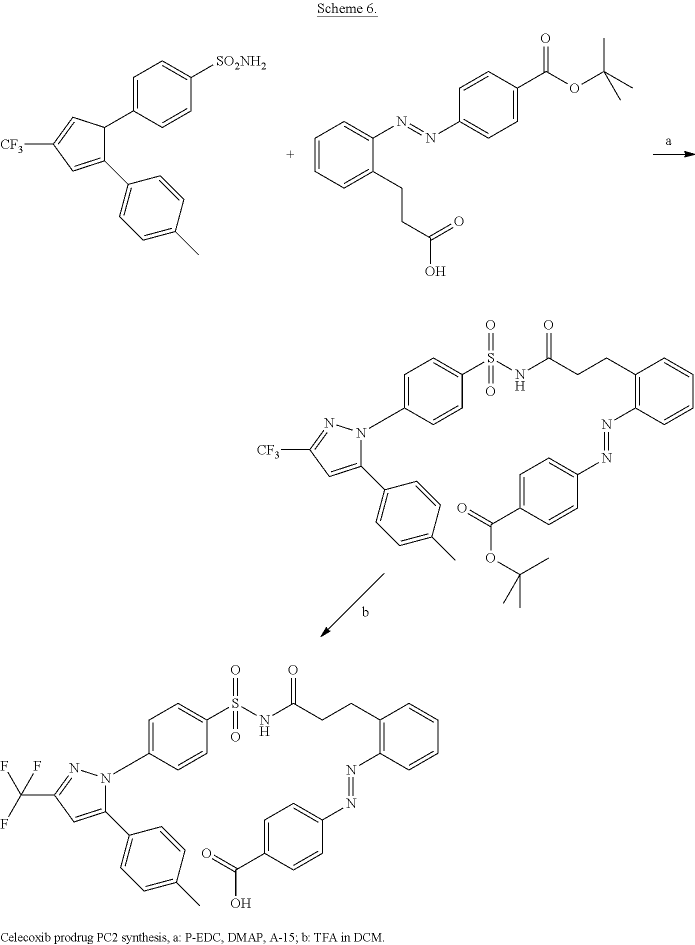

### Experimental Procedure

### (E)-tert-butyl 4-((2-(3-oxo-3-(2-(p-tolyl)-4-trifluoromethyl)cyclopenta-2,4-dien-1-yl)phenylsulfonamido)propyl)phenyl)diazenyl)benzoate

Into a round bottom flask was added the azo carrier (0.21 mmol, 0.076 g), t-BuOH (3 ml), ClCH₂CH₂Cl (3 ml), DMAP (3 eq, 0.64 mmol, 0.078 g), P-EDC (1.03 g) and the celecoxib (0.7 eq, 0.15 mmol, 0.06 g). The resulting mixture was allowed to stir at room temperature for 24 hours. The reaction mixture was then diluted with EtOAc (4 ml), and Amberlyst-15 (1.58 g) was added to the reaction and the reaction was left stirring for additional two hours. At this time the reaction was filtered through a short plug of silica gel and the residue was rinsed with additional EtOAc. The resulting filtrate was concentrated to afford the product as orange oil (0.1 g). The crude mixture was columned using DCM:EtOAc (9:1) to yield the product as orange oil (0.07 g, 46.4%). ¹H-NMR (CDCl₃) δ: 8.13 (d, 2H, J=8.24 Hz), 7.95 (d, 2H, J=8.56 Hz), 7.87 (d, 2H, J=8.52 Hz), 7.68 (d, 1H, J=7.52 Hz), 7.44 (d, 2H, J=8.04 Hz), 7.34 (t, 2H, J=8.04 Hz), 7.27 (t, 2H, J=7.28 Hz), 7.18 (d, 2H, J=7.28 Hz), 7.12 (d, 2H, J=8.04 Hz), 6.76 (s, 1H), 3.41 (t, 2H, J=7.28 Hz), 2.70 (t, 2H, J=7.52 Hz), 2.38 (s, 3H), 1.63 (s, 9H). ¹³C-NMR (CDCl₃) δ: 165.90, 165.13, 154.85, 149.85, 145.30, 144.76, 143.58, 142.39, 139.86, 137.89, 137.27, 132.17, 130.63, 130.52, 129.81, 129.34, 128.77, 128.65, 128.35, 127.65, 125.65, 125.05, 122.63, 118.23, 115.72, 113.73, 106.50, 81.62, 38.56, 28.11, 26.36, 21.34.

### (E)-tert-butyl 4-((2-(3-oxo-3-(2-(p-tolyl)-4-trifluoromethyl)cyclopenta-2,4-dien-1-yl)phenylsulfonamido)propyl)phenyl)diazenyl)benzoic acid

The tert-butyl ester of the PC2 (0.07 g) was dissolved in DCM/TFA 1:1 (2 ml) at room temperature for four hours, until the TLC analysis (EtOAc) showed reaction completion The solvent was blow off with nitrogen and the crude mixture was columned to yield the product as a orange crystals (0.05 g, 78%). ¹H-NMR (MeOD) δ: 8.17 (d, 2H, J=8.56 Hz), 7.91 (t, 5H, J=9.52 Hz), 7.66 (d, 1H, J=7 Hz), 7.44 (d, 2H, J=9.04 Hz), 7.34 (t, 2H, 6.52 Hz), 7.29 (m, 2H), 7.15 (m, 3H), 6.91 (s, 1H), 3.39 (t, 2H, J=7.52 Hz), 2.64 (t, 2H, J=7.52 Hz), 2.28 (s, 3H). ¹³C-NMR (MeOD) δ: 165.78, 165.32, 154.21, 149.37, 145.18, 141.76, 140.77, 140.58, 139.10, 137.93, 137.34, 132.57, 131.18, 130.85, 129.96, 129.77, 128.77, 128.70, 127.81, 126.38, 125.27, 124.69, 121.72, 117.56, 115.31, 114.42, 105.10, 38.73, 26.22, 19.41. HRMS: Expected (M⁺)=662.1679, Found (M⁺)=662.0872.

Efficacy

The general approaches and background are described in the background to the invention. The efficacy of the compounds of the present application may be demonstrated in mouse models of colorectal cancer. Colorectal cancer arises from a well characterized sequence of mutations. Numerous genetically engineered mouse strains are available for studying the progression of colorectal disease and these can be applied to testing compounds for treatment and chemoprevention.

Selective delivery of COX-2 inhibitors to the colon is expected to attenuate or abrogate the development of lesions in the Min or APCMin/+, model. The chemopreventative and therapeutic effects of the compounds may also be demonstrated in rodent models of colorectal cancer where tumors are chemically induced using agents such as Azoxymethane (AOM). AOM is a potent carcinogen used to induce aberrant crypt foci (ACFs) in mice and rats. It may be administered to rats at (20 mg/kg) by subcutaneous injection. AOM is useful in evaluating efficacy of preventative treatment for carcinogenesis where COX-2 expression is known to play a prominent role in disease progression. The compounds may be given chronically by the oral route to rats before or after insult with AOM. The effect of the prodrug administration on disease progression can be followed by scoring for number and size of tumors relative to vehicle and/or oral COX-2 treatment at the same dose.

Intramolecular Lactamization of the Celecoxib Prodrug (PC1) Intermediate at Different pH Values

The following data and accompanying graphs show the cyclization and drug release from the amino compound by bacterial reduction. The intermediate synthesised cyclizes at different rates depending on the pH. The pH of the colon ranges from around 4 to 8, and the following data show that under these conditions lacamization is sufficiently fast for clinically relevant rates of drug release. Lactamization is especially rapid in the pH range 4-6. The rapid kinetics of the aminolysis of sulfonamides is unexpected and surprising, as is its high pH dependence, as evidenced by the slow lactamization at pH>8.

### Experimental

To test the intramolecular lactamization the reduced intermediate of the celecoxib prodrug PC1 was chemically synthesised. The cyclization kinetics for the celecoxib prodrug was evaluated at different pH values from 2.6 to 8. Lactamization studies were carried out in borate buffer solution with the ionic strength of 0.12 at 37° C.

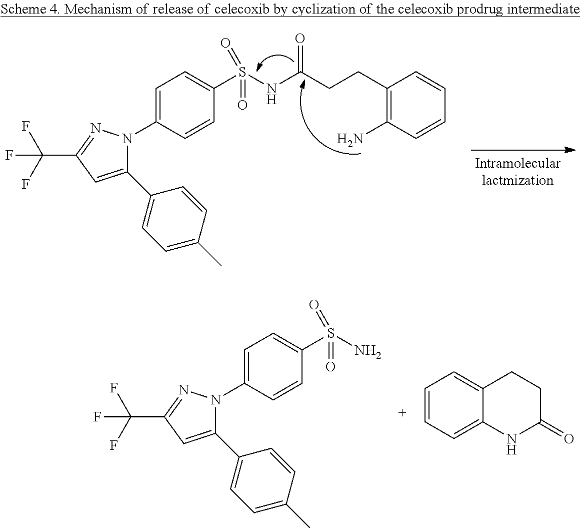

Synthesis of the PC1 Intermediate:

the intermediate was prepared BOC-protected because otherwise it would undergo spontaneous lactamization. The BOC group is taken off immediately before starting the lactamization experiment:

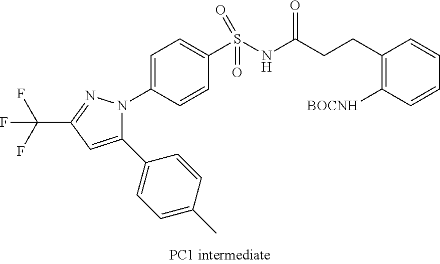

### (2-{3-Oxo-3[4-5(-p-tolyl-3-trifluoromethyl-pyrazol-1-yl)-benzenesulfonylamino]-propyl}-phenyl)-carbamic acid tert-butyl ester

Into a round bottom flask was BOC-amino phenyl propionic acid (0.78 mmol, 0.2 g), t-BuOH (9 mL), ClCH₂CH₂Cl (9 mL), DMAP (3 eq, 2.34 mmol, 0.27 g), P-EDC (3.6 g) and the Celecoxib (0.7 eq, 0.54 mmol, 0.21 g). The reaction mixture was then diluted with EtOAc (4 ml), and Amberlyst-15 (4.8 g) was added to the reaction and the reaction was left stirring for additional two hours. At this time the reaction was filtered through a short plug of silica gel and the residue was rinsed with additional EtOAc. The resulting filtrate was concentrated to afford the product as colourless oil (0.26 g). The crude mixture was columned using Hexane:EtOAc (7:3) as a mobile phase to yield the product as white off crystals (0.19 g, 60%) m.p 184-186° C. IRᵥₘₐₓ(KBr): 3437.21, 1564.30, 1415.85, 1261.62 cm⁻¹, ¹H-NMR (MeOD) δ: 9.48 (s, 1H, NH), 7.89 (d, 2H, J=8.52 Hz), 7.45 (d, 2H J 9.04 Hz), 7.37 (d, 1H, J=7.56 Hz), 7.21 (d, 3H J 8.04 Hz), 7.14 (d, 2H, J=8.04 Hz), 6.96 (t, 1H, J=7.52 Hz), 6.92 (d, 1H, J=6.52 Hz), 6.5 (s, 1H, NH), 2.84 (t, 2H, J=7 Hz), 2.48 (t, 2H J 7.04 Hz), 2.40 (s, 3H), 1.53 (s, 9H). ¹³C-NMR (MeOD) δ: 178.64 (C, C═O, C-1), 155.13 (C═O, C-10), 145.48 (C, C-21), 143.87 (C, C-19), 141.05 (C, C-16), 139.44 (C, C-25), 135.59 (C, C-13), 134.96 (C, C-5), 132.19 (C, C-4), 131.01 (C, C-22), 129.22 (CH, C-26, C-24), 129.19 (CH, C-23, C-27), 128.62 (CH, C-14, C-18), 127.80 (CH, C-7, C-9), 125.98 (CH, C-8), 124.89 (C, C-29), 124.82 (CH, C-6), 124.65 (CH, C-17, C-15), 105.29 (CH, C-20), 79.46 (C, C-11), 39.44 (CH₂, C-3), 27.34 (CH₃, C-12), 26.60 (CH₂, C-3), 22.34 (CH₃, C-28). HRMS: Expected (M-Na⁺)=651.1875, Found (M-Na⁺)=651.11865.

Lactamization Data

Lactamization in acidic solution was completed quickly. The higher the pH the slower the lactamization; however in all the cases the lactamization was fast enough to suggest release celecoxib within the orocecal transit time. The following Table 1 provides data at different pHs, and FIGS. 1-3 of the accompanying drawings show in graphic form the lactamization profile at:

FIG. 1: pH 5.52,

FIG. 2: pH 6.4 and

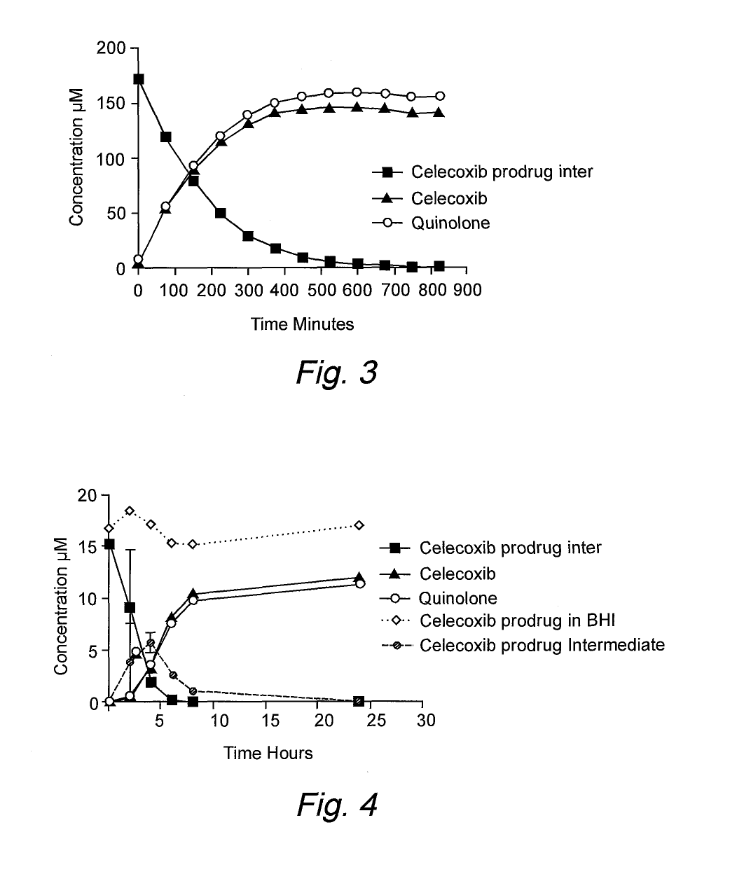

FIG. 3: pH 7.

TABLE 1
Celecoxib prodrug kinetic data

| pH | K_{obs} | Half life min | SEM |
|---|---|---|---|
| 7.4 | 0.001097 | 631.8 | 0.00007 |
| 7 | 0.005314 | 130.40 | 0.0004 |
| 6.4 | 0.01150 | 60.28 | 0.0002 |
| 6.28 | 0.01682 | 41.22 | 0.0002 |
| 6.2 | 0.02347 | 29.53 | 0.0003 |
| 6.11 | 0.04553 | 15.23 | 0.004 |
| 6.0 | 0.06329 | 10.95 | 0.016 |
| 5.52 | 0.06823 | 10.16 | 0.022 |

In Vitro Reduction and Lactamization in the Presence of Clostridium perfringens

The following confirms that the presence of Clostridium perfringens found in the colon can catalyse the in vitro reduction and lactamization of the azo group of PC1 revealing an amine which spontaneously cyclises (as described above). The pH involved was not optimal for lactamization, but the drug release was still within an acceptable timeframe.

Method

The azoreductase activity of Clostridium perfringens towards PC1 was tested under anaerobic conditions at 37° C. The bacteria were grown in BHI and the initial concentration of prodrug was 50 μM. Aliquots were withdrawn at appropriate time intervals and quenched using two volumes of acetonitrile and centrifuged for 10 min at 10000 rpm.

Stock solution of PC1 was prepared in DMSO at concentration of 5 mM.

Culture of Clostridium perfringens

Clostridium perfringens were inoculated on agar plates containing BHI media. The next day, Clostridium perfringens were scratched from the agar plates and inoculated in BHI media to obtain cell density between 0.9 and 1.1 at 600 nm, into bacteria suspension (1980 μL) in sterile 20 mL universal tube was added 20 μl of prodrug stock solution to reach 50 μM concentration and 1% DMSO, the negative control consisted of BHI media with test prodrugs at the same concentration.

Immediately after prodrug was added to the cells, an aliquot was taken (2000 and added to 400 μl of acetonitrile in 1.5 ml test tube; samples were then centrifuged at 10000 rpm for 10 min.

Supernatant was transferred to a tube and stored at −20° C. until analysis, aliquots were taken every 2 hours and time points covered were: 2, 4, 6, 8 and 24 h.

Extraction of PC1 and Celecoxib from BHI

The following tables confirm the prodrug reduction and release of active drug. Extraction of PC1 and celecoxib from BHI media using two volumes of acetonitrile was carried out in order to establish if we were able to get accurate data from the reduction experiment using Clostridium perfringes.

TABLE 2
PC1 extraction from BHI media

| | | | |
|---|---|---|---|
| | Average concentration | % Concentration | |
| PC1 | recovered [μM], n#2 | recovered | RSD |

| [μM] | Exp1 | Exp2 | Exp1 | Exp2 | % |
|---|---|---|---|---|---|
| 1.1 | 1.4 | 1.1 | 131.0 | 101.4 | 18.0 |
| 5.5 | 6.0 | 5.9 | 109.0 | 105.8 | 2.1 |
| 16.6 | 17.1 | 18.4 | 102.8 | 110.8 | 5.3 |

TABLE 3
Celecoxib extraction from BHI media

| | Average concentration | | % Concentration | | |
|---|---|---|---|---|---|
| Celecoxib | recovered [μg/ml], n#2 | | recovered | | RSD |
| [μM] | Exp1 | Exp2 | Exp1 | Exp2 | % |
| 1.1 | 1.1 | 1.1 | 97.3 | 102.8 | 3.9 |
| 5.5 | 5.1 | 5.1 | 93.1 | 93.2 | 0.1 |
| 16.6 | 16.0 | 16.9 | 96.5 | 102.0 | 4.0 |

Results of Azoreductase/Cyclization Using Clostridia Azoreductase activity was observed for PC1, as shown in Table 4 below and graphically in FIG. 4 of the accompanying drawings.

TABLE 4
Azoreductase activity of Clostridium perfringens towards PC1

| Time | Celecoxib | | Celecoxib | | Quinolone | | Celecoxib prodrug | | Celecoxib prodrug |
|---|---|---|---|---|---|---|---|---|---|
| Hours | prodrug μM | | μM | | μM | | intermediate μM | | in BHI μM |
| 0 | 15.59 | 14.97 | 0.00 | 0.00 | 0.00 | 0.00 | 0.00 | 0.00 | 16.70 |
| 2 | 3.57 | 14.68 | 0.66 | 0.39 | 0.67 | 0.00 | 7.57 | 0.13 | 18.40 |
| 4 | 0.00 | 3.82 | 3.56 | 3.09 | 3.61 | 3.12 | 6.76 | 4.79 | 17.14 |
| 6 | 0.00 | 0.36 | 8.3 | 7.96 | 7.8 | 7.40 | 2.94 | 2.25 | 15.29 |
| 8 | 0.00 | 0.00 | 10.08 | 10.80 | 9.51 | 9.97 | 1.25 | 0.94 | 15.15 |
| 24 | 0.00 | 0.00 | 11.64 | 12.34 | 11.16 | 11.56 | 0.00 | 0.00 | 16.99 |

### CONCLUSION

The above confirms the ability of the present invention to provide compounds and pharmaceutical compositions that may be used for the treatment and prevention of gastrointestinal diseases including, but not limited to, cancers such as colorectal and bowel cancer, and inflammatory diseases such as Inflammatory Bowel Disease (IBD). The compounds are prodrugs of sulfonamide drugs, particularly but not exclusively COX-2 inhibitors in which the parent COX-2 inhibitor is linked to a carrier.

The prodrugs may be taken orally, and are designed so that they are not absorbed into the blood stream from the oral route. Instead the inhibitors transit to the colon where they are acted upon by colonic microflora releasing the COX-2 or other inhibitor. In one aspect of the design of the present application, the group attached to the COX-2 or other inhibitor (generating the prodrug) is itself also chemopreventative after activation in the colon. For example, the coupling of celecoxib to 5-ASA, which is a colonic anti-inflammatory that reduces the risk of cancer. The combination is designed to be activated by colonic microflora with complementary and potentially synergistic effects in cancer prevention.

Safety

The cardiovascular risk associated with COX-2 inhibition is due to systemic availability of the compounds and their effect on the production of prostacyclin by the cardiovascular endothelium. The compounds of the present application are safer because they restrict COX-2 inhibitor release to the colon. This may be demonstrated pharmacokinetically using standard methods known in the art and as cited herein. Oral administration of the prodrug compounds is expected to be associated with increased fecal excretion of the parent COX-2 inhibitors (valdecoxib or celecoxib, for example) and lower peripheral levels of the inhibitors. This may be shown using high performance liquid chromatography (preferably with MS detection) to measure the fecal, urinary and plasma levels of the parent following administration of either the prodrug or the parent.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

The entire disclosures of all documents cited throughout this application are incorporated herein by reference.

1. A compound of the formula I: 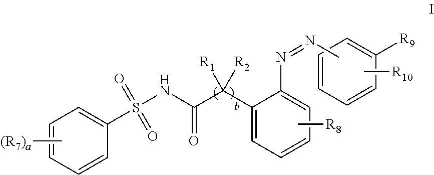
wherein:
each R₁ and R₂ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each alkyl, cycloalkyl, aryl and heteroaryl substituted or unsubstituted; or R₁ and R₂ are together an oxo (═O) group;
each R₃ and R₄ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each alkyl, cycloalkyl, aryl and heteroaryl substituted or unsubstituted; or R₃ and R₄ are together an oxo (═O) group;
each R₅ and R₆ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each alkyl, cycloalkyl, aryl and heteroaryl substituted or unsubstituted; or R₅ and R₆ are together an oxo (═O) group;
R₇ is selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, heteroarylaryl, arylheteroaryl, (C₃₋₁₀)heterocyclyl, amino, carboxy, cyano, halo, hydroxy, sulfamoyl, CONR₁₁R₁₂, (C₁₋₃)alkoxy, aryloxy and heteroaryloxy, each alkyl, cycloalkyl, aryl and heteroaryl substituted or unsubstituted; or alternatively two of R₇, when substituted adjacent on the phenyl ring, together form an aryl, heteroaryl, (C₃₋₁₀)cycloalkyl or (C₃₋₁₀)heterocycloalkyl ring, each substituted or unsubstituted;
each R₈ and R₁₀ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, —SO₃R₁₃, —PO₃R₁₃, (C₁₋₃)alkoxy, aryloxy and heteroaryloxy, each alkyl, cycloalkyl, aryl and heteroaryl substituted or unsubstituted;
R₉ is selected from the group consisting of hydrogen, hydroxy, (C₁₋₃)alkoxy, and —CO₂R₁₃;
or R₉ and R₁₀, when substituted adjacent in the phenyl ring, are taken together to form an optionally substituted heterocyclic ring;
each R₁₁ and R₁₂ is independently selected from the group consisting of hydrogen, (C₁₋₃)alkyl and aryl;
R₁₃ is hydrogen or (C₁₋₃)alkyl;
each a and b is independently 0, 1, 2 or 3;
or a pharmaceutically acceptable salt thereof, optionally in the form of a single stereoisomer or mixture of stereoisomers.

2. A compound of the formula II: 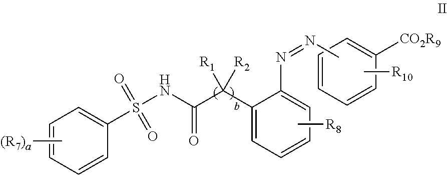
wherein:
R₁₋₈ are as defined in claim 1;
each R₉ and R₁₀ is independently selected from the group consisting of hydrogen, and (C₁₋₃)alkyl;
R₁₁₋₁₃ and a and b are defined in claim 1;
or a pharmaceutically acceptable salt thereof, optionally in the form of a single stereoisomer or mixture of stereoisomers.

3. The compound of claim 1 or 2, wherein b is 1 or 2.

4. The compound of claim 1 or 2, wherein R₉ and R₁₀ are hydrogen.

5. A compound of the formula III: 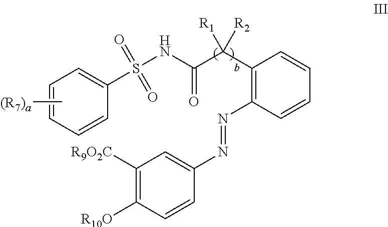
wherein:
each R₁, R₂, R₇, R₉, R₁₀, a and b are as defined in any one of claims 1-4; or a pharmaceutically acceptable salt thereof, optionally in the form of a single stereoisomer or mixture of stereoisomers.

6. The compound of claim 5, wherein R₁ and R₂ are hydrogen.

7. The compound of claim 5, wherein R₁, R₂, R₉ and R₁₀ each are hydrogen.

8. The compound of claim 1, wherein the compound is selected from the group consisting of: 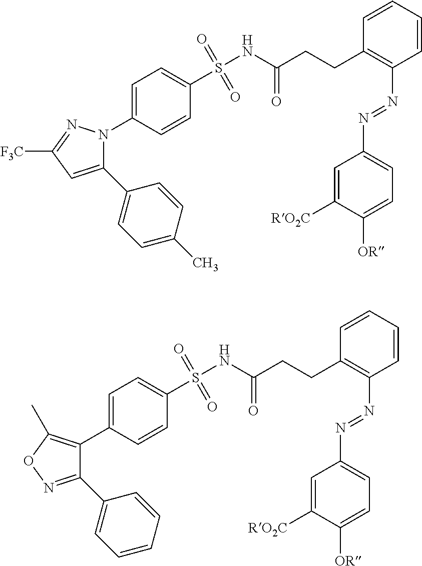
wherein:
R′ is H or (C₁₋₃)alkyl; and
R″ is H or (C₁₋₃)alkyl.

9. The compound of claim 1 being a prodrug wherein the (R₇)ₐ-phenyl-S(O)₂NH group represents a sulfonamide-bonding compound.

10. The compound of claim 9 wherein the sulfonamide-bonding compound is ethoxzolamide.

11. A pharmaceutical composition comprising a therapeutically effective amount of a compound of claim 1, and a pharmaceutically acceptable excipient.

12. A compound or composition of claim 1 for use in therapy.

13. A compound or composition of claim 1 for inhibiting COX-2 activity.

14. A compound or composition of claim 1 for treating cancer and various gastrointestinal diseases, including inflammatory bowel disease (IBD) and colorectal cancer.

15. A method for inhibiting COX-2 activity in a patient, the method comprising administering a therapeutically effective amount of a compound or composition of claim 1 to the patient.

16. The method of claim 15, wherein the therapeutically effective amount is effective to reduce, alleviate, treat or prevent the development of colorectal cancer.

17. The method of claim 15, wherein the patient has an increased genetic risk of cancer.

18. The method of claim 15, wherein the amount of a compound or composition administered is effective to maintain remission.

19. The method as claimed in any one of claims 15 to 18 comprising the co-administration sequentially, simultaneously and/or separately of a therapeutically effective amount of one or more other compounds or compositions able to reduce, alleviate, treat or prevent the development of cancer and/or various gastrointestinal diseases, including inflammatory bowel disease (IBD) and colorectal cancer.

20. The method of claim 19 wherein the one or more other compounds or compositions comprises one or more from the group comprising; atorvastatin, valdecoxib, erlotinib and celecoxib.

21. A method for treating gastrointestinal cancer in a mammal, the method comprising delivering a therapeutically effective amount of a COX-2 inhibitor to the colon, wherein the COX-2 inhibitor is released in-vivo from the composition of claim 11.
